(19) United States
(12) Patent Application Publication
(10) Pub. No.: US 2025/0000232 A1
(43) Pub. Date: Jan. 2, 2025

(54) APPLICATOR WITH SKIN COOLING SYSTEM

(71) Applicant: L'Oreal, Paris (FR)

(72) Inventors: Rafael FELICIANO, New Providence, NJ (US); Hyosik Dennis MIN, Palisades Park, NJ (US)

(73) Assignee: L'Oreal, Paris (FR)

(21) Appl. No.: 18/345,084

(22) Filed: Jun. 30, 2023

(51) Int. Cl. A45D 34/04

(52) U.S. Cl. CPC A45D 34/041; A45D 2034/002; A45D 2200/055; A45D 2200/155

(57) ABSTRACT

An applicator including a position and formula deposition component configured to sense position measurands, and delivery measurands and to generate map data and treatment area data of a skin area as an applicator surface applies one or more formulas to the skin area; and a cooling element operably coupled to the position and formula deposition component, the cooling element configured to apply a cooling treatment to one or more treated areas responsive to one or more inputs from the position and formula deposition component wherein the one or more treated areas are one or more areas of the skin where the one or more formulas have been applied.

### SUMMARY

In one aspect, disclosed herein is an applicator, including a body, an applicator surface configured to apply one or more formulas to skin, one or more reservoirs configured to hold the one or more formulas, a position sensor configured to map the skin as the applicator surface applies the one or more formulas, a formula sensor configured to detect one or more treated areas of the skin, wherein the one or more treated areas comprise one or more areas of the skin where the one or more formulas have been applied, and a cooling element configured to apply a cooling treatment to the one or more treated areas.

In some embodiments, the cooling element includes a Peltier plate. In some embodiments, the cooling element includes a pump configured to pump air, a compression chamber coupled to the pump, where the compression chamber is configured to move the air to the applicator surface, a solenoid configured to cool the air as it moves to the applicator surface, and an outlet configured to apply the air to the skin.

In some embodiments, the applicator is configured to direct the cooling element to apply the cooling treatment only to the one or more treated areas.

In some embodiments, the applicator surface comprises one or more nozzles, wherein the one or more nozzles are in fluid communication with the one or more reservoirs. In some embodiments, each nozzle of the one or more nozzles is configured to dispense a different formula of the one or more formulas.

In some embodiments, the applicator surface is a roller ball, wherein the roller ball is in fluid communication with the one or more reservoirs and the roller ball is configured to apply the one or more formulas as the roller ball rolls. In some embodiments, the applicator further comprises a groove located around the roller ball. In some embodiments, the cooling element is configured to apply the cooling treatment from the groove on the applicator.

In another aspect, disclosed herein is a system for applying a cooling treatment, the system including the applicator described herein, and a dispensing device configured to couple to the applicator, where the dispensing device includes a dispenser processor configured to direct the applicator to apply the one or more formulas.

In some embodiments, the applicator surface is a roller ball, where the roller ball is in fluid communication with the one or more reservoirs and the roller ball is configured to apply the one or more formulas as the roller ball rolls. In some embodiments, the applicator further comprises a groove located around the roller ball. In some embodiments, the cooling element is configured to apply the cooling treatment from the groove on the applicator.

In some embodiments, the cooling element includes a Peltier plate. In some embodiments, the cooling element includes a pump configured to pump air, a compression chamber coupled to the pump, where the compression chamber is configured to move the air to the applicator surface, a solenoid configured to cool the air as it moves to the applicator surface, and an outlet configured to apply the air to the skin.

In some embodiments, the formula sensor is further configured to transmit treatment area data to the dispenser processor, and wherein the dispenser processor is further configured to direct the applicator to apply a cooling treatment to the one or more treated areas.

In some embodiments, the system further includes a communication device, configured to visualize the skin, highlight one or more treated areas of the skin, where the one or more treated areas of the skin are areas on the skin where a user has applied the one or more formulas, locations based on a user history, or a combination thereof, direct the user to move the applicator over the one or more treated areas, and store the user history, wherein the user history is each time, location, or a combination thereof that the user applies the cooling treatment, the formula, or a combination thereof.

In yet another aspect, disclosed herein is a method of applying a cooling treatment with the system as described herein, the method including applying one or more formulas to skin, detecting one or more treated areas, where the one or more treated areas comprise areas of the skin where the one or more formulas have been applied, and applying a cooling treatment to the one or more treated areas with the cooling element.

In some embodiments, the method further includes rolling a roller ball of the applicator surface on the skin to apply the one or more formulas to the skin, and applying the cooling treatment from a groove around the roller ball.

In some embodiments, the method further includes visualizing the skin with a communication device, highlighting one or more treated areas of the skin, wherein the one or more treated areas of the skin are areas on the skin where a user has applied the one or more formulas, locations based on a user history, or a combination thereof, directing the user to move the applicator over the one or more treated areas, and storing the user history, wherein the user history is each time, location, or a combination thereof that the user applies the cooling treatment, the formula, or a combination thereof.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is an example applicator, in accordance with the present technology;

FIG. 1B is a cross section of the applicator of FIG. 1A, in accordance with the present technology;

FIG. 1C is an example cooling element of the applicator of FIG. 1A, in accordance with the present technology;

FIG. 1D is a top view of the applicator of FIG. 1A, in accordance with the present technology;

FIG. 2A is another example applicator, in accordance with the present technology;

FIG. 2B is a cross section of the applicator of FIG. 2A, in accordance with the present technology;

FIG. 3 is an example system, in accordance with the present technology;

FIG. 4 is another example system, in accordance with the present technology;

FIG. 5A is an example cooling element in use, in accordance with the present technology;

FIG. 5B is another example cooling element in use, in accordance with the present technology;

FIG. 6 is an example method of using a system, in accordance with the present technology; and

FIG. 7 is another example method of using a system, in accordance with the present technology.

### DETAILED DESCRIPTION

Disclosed herein are applicators, systems, and methods for applying a one or more formulations to the skin, and then applying a localized cooling treatment to the skin, where the formula was applied.

In some embodiments, the applicators include an application surface configured to apply the one or more formulas, and to apply the cooling treatment. The applicator surface can take a number of forms, including a roller ball and a flat applicator surface, each configured to apply the one or more formulas. The applicator may further include one or more sensors, including position and/or condition sensors, one or more reservoirs configured to hold one or more formulas, and a cooling element.

The cooling element may take any number of forms. In some embodiments, the cooling formula is a Peltier plate. In other embodiments, the cooling element may be a solenoid cooling elements, configured to direct cold air towards the skin. In some embodiments, such as when the applicator surface is a roller ball, the cooling element may be configured to blow cold air through a groove around the roller ball.

In some embodiments, the one or more formulas are skin care formulas. In some embodiments, the skin care formulas include a moisturizer, a toner, an acne treatment, a wrinkle treatment, fine line treatment, eye cream, or a cosmetic. In some embodiments, the one or more formulas are one or more ingredients of a skin care formula. In some embodiments, at least one of the one or more formulas is a cooling formula, such as capsaicin, aloe, caffeine, or the like.

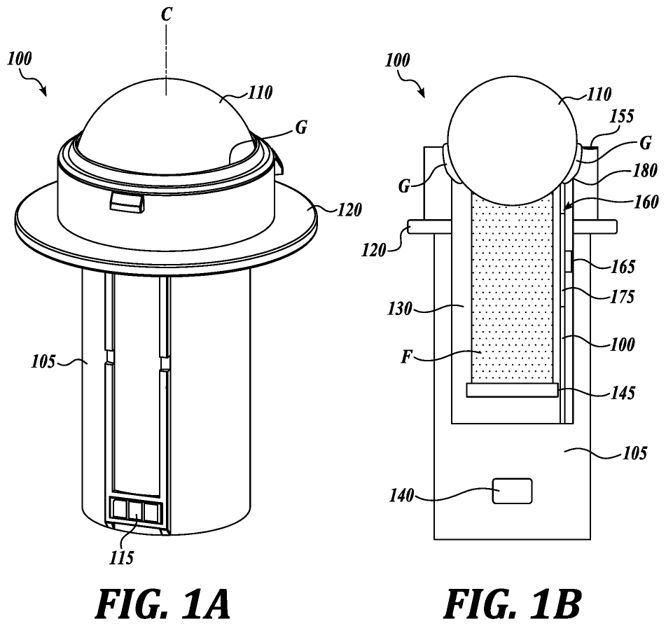

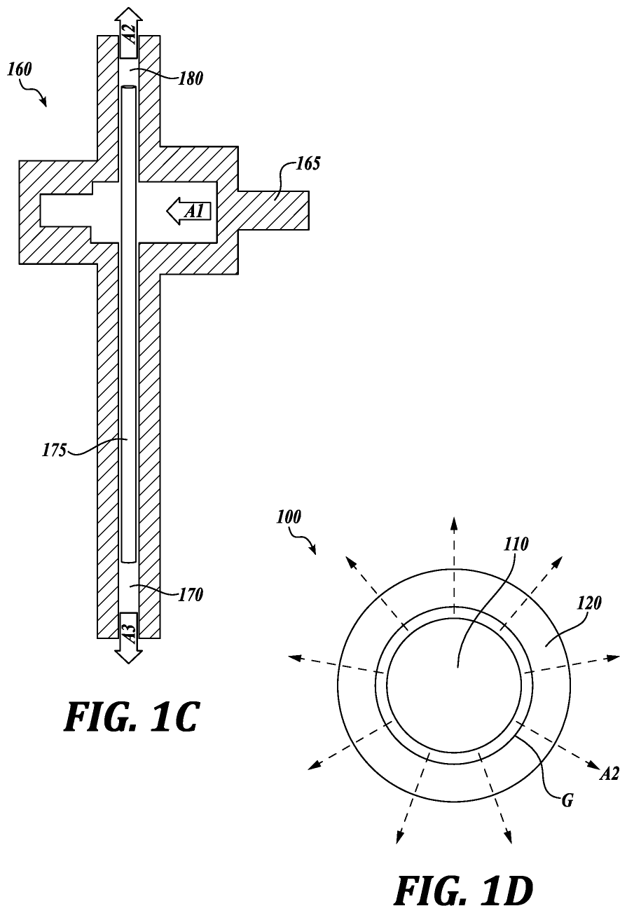

FIG. 1A is an example applicator 100, in accordance with the present technology. The applicator 100 may include a body 105, an applicator surface 110, a tag 115, and an attachment 120. Also illustrated is a cross-section line C.

In some embodiments, the applicator 100 includes a body 105. In some embodiments, the body 105 is configured to contain additional components and/or circuitry, as shown in FIGS. 1B-1D.

The applicator surface 110 may take any number of forms, including a roller ball configured to distribute and apply a formula located a reservoir inside the applicator 100 (as shown in FIGS. 1A-1D). In some embodiments, the roller ball 110 is plastic, but in other embodiments, the roller ball 110 may be glass or metal.

In some embodiments, there is a grove G disposed between the applicator surface 110 and the applicator 100. In some embodiments, the groove G is configured to provide a cooling treatment, as described in detail herein.

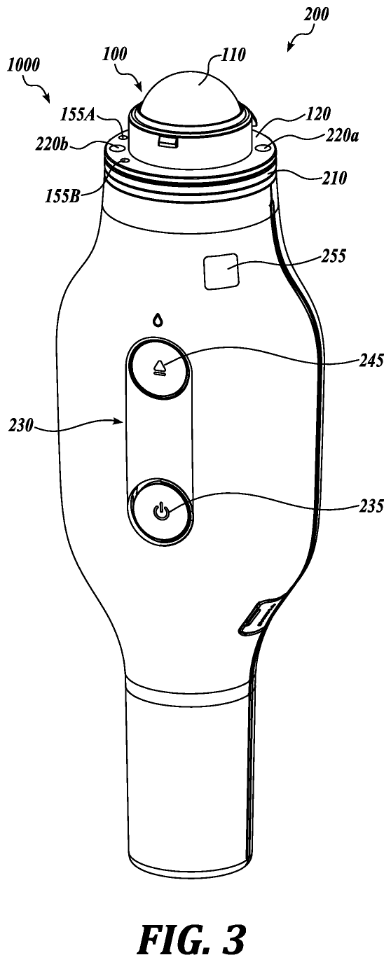

In some embodiments, the applicator 100 further includes a tag 115. In some embodiments, the tag 115 is a QR code, RFID tag, barcode, or the like. In some embodiments, the tag 115 communicates an identity of the applicator 100 or the one or more formulas to a dispensing device, such as shown in FIG. 3. In some embodiments, the tag 115 may be used to identify any number of things about the one or more formulas or applicator 100, including the amount of formula inside the applicator 100, the expiration date of the formula 140 inside the applicator 100, or when to replace the applicator 100.

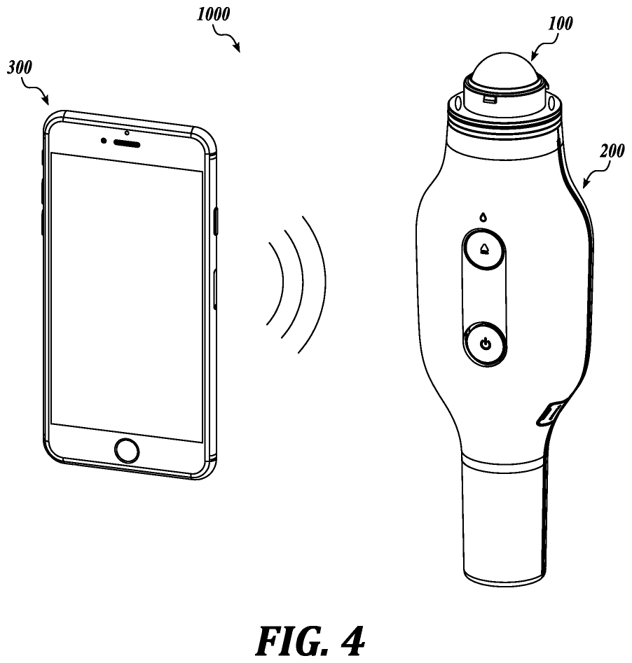

In some embodiments, the applicator 100 also includes an attachment 120 configured to secure the applicator 100 into a dispensing device, such as the dispensing device 200 in FIGS. 3-4. While the attachment 120 is illustrated as a disk shaped to couple to a dispensing device, the attachment 120 may take any form capable of securing the applicator to a dispensing device including a threaded attachment, a magnet, or an attachment configured to snap into the dispensing device. In some embodiments, the attachment 120 is clear so that the dispensing device is visible through the attachment. In some embodiments, the applicator 100 is configured to apply cooling treatment.

In operation, the applicator 100 can be placed inside a dispensing device (as shown in FIG. 3) and secured to the dispensing device with the attachment 120. The roller ball 110 can be rolled over a surface, such as a user's skin, to apply one or more formulas. A cooling treatment may then be applied to the areas that the one or more formulas have been applied, from groove G.

FIG. 1B is a cross section of the applicator of FIG. 1A, in accordance with the present technology. FIG. 1B is a view taken from the cross-section line C of FIG. 1A. The applicator 100 may include a body 105, an applicator surface 110, an attachment 120, a reservoir 130 configured to hold a formula F, a piston 145, and an applicator processor 140. In some embodiments, the applicator 100 further includes a cooling element 160, as shown and described in further detail in FIG. 1C. In some embodiments, the cooling element 160 includes a pump 165, a cooling chamber 170, a solenoid 175, and an outlet 180.

In some embodiments, the reservoir 130 is located inside the body 105, and is configured to hold a formula F. In some embodiments, the formula F is a skin care formula. In some embodiments, the skin care formula is a moisturizer, a toner, an acne treatment, a wrinkle treatment, fine line treatment, eye cream, or a cosmetic, such as a foundation or concealer. As the applicator surface 110, illustrated here as a roller ball, moves or rolls, formula F from the reservoir 130 is applied to a surface.

In some embodiments, the applicator 100 further includes a piston 145 configured to push the formula 140 towards the roller ball 110 as the formula is applied. In some embodiments, the piston 145 is directed by circuitry on a dispensing device or on the applicator 100 itself to push the formula 140 towards the applicator surface 110.

In some embodiments, the applicator 100 includes an applicator processor 140. In some embodiments, the applicator processor 140 is configured to direct the piston to push the formula 140 towards the applicator surface. In some embodiments, the applicator processor 140 is further configured to direct the applicator to supply a cooling treatment with the cooling element 160.

FIG. 1C is an example cooling element 160 of the applicator 100 of FIG. 1B, in accordance with the present technology. In some embodiments, the cooling element 160 includes a pump 165, a cooling chamber 170, a solenoid 175, and an outlet 180. In some embodiments, the pump 165 is a compressor, such as an air compressor. In some embodiments, the cooling chamber 170 is a tube within the body (such as body 105) in an applicator (such as applicator 100). In some embodiments, the cooling chamber 170 is made of metal or glass to allow the cooling chamber 170 to become colder, faster. In some embodiments, a solenoid 175 is disposed within the cooling chamber 170.

In operation, air A1 flows from the pump 165 and into the cooling chamber 170. In some embodiments, the air moves across the solenoid 175 and separates into cold air A2 and warm air A3. In some embodiments, the cold air A2 flows out of the outlet 180. The cold air A2 may then flow out of the applicator 100 as cooling treatment, as shown in FIG. 1D. The warm air A3 moves downward, and out of the base of the applicator.

In some embodiments, the cooling element 160 is a vortex tube, or a Ranque-Hilsh vortex tube. In some embodiments, the vortex tube separated a compressed gas, from pump 165, into hot air A3 and cold air A2 streams. In some embodiments, a gas is injected into cooling chamber 170, leading to rapid rotation (or a first vortex) as it moves along an inner surface of the cooling chamber 170 towards a far end (where hot air A3 is shown exiting the cooling chamber). The outlet 180 allows the gas from an outer layer of the first vortex to escape through the outlet 180. In some embodiments, the outlet 180 is a conical nozzle. The remainder of the gas may then be forced to return in an inner vortex with a smaller diameter than the first vortex. Gas from the inner vortex transfers heat to the gas in the first vortex so that the outer layer of the first vortex is hotter at the far end than before. The gas in the inner vortex may likewise be cooler upon its return to the outlet 180, where it is released from the cooling chamber 170. In this manner, it is the outlet 180 that provides the cooling treatment A2.

FIG. 1D is a top view of the applicator 100 of FIG. 1A, in accordance with the present technology. In some embodiments, the cool air A2 of FIG. 1C flows from the outlet of the cooling element (such as outlet 180) and through the groove G in the applicator 100. In some embodiments, the groove G is disposed around the applicator surface 110. In some embodiments, the groove G is configured to administer cooling treatment in the form of cool air A2 in 360 degrees from the applicator surface 110. In some embodiments, the cool air A2 (also referred to as the cooling treatment herein) is directed to flow from only a portion of the groove G. For example, in some embodiments, the cooling element is configured to administer cooling treatment A2 in the direction that the applicator 100 is being moved about the skin.

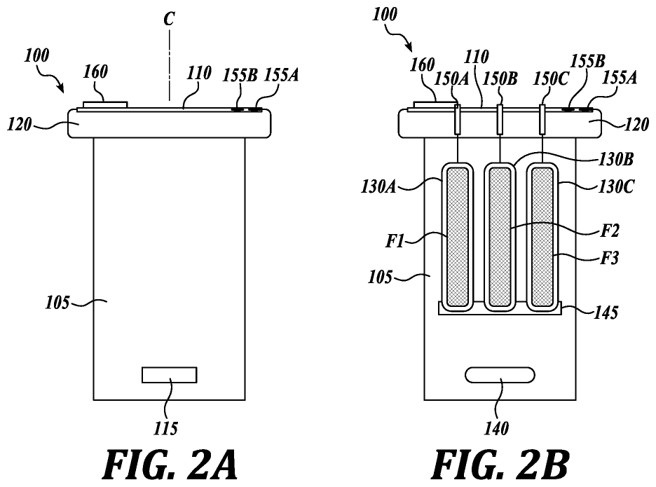

FIG. 2A is another example applicator 100, in accordance with the present technology. In some embodiments, the applicator 100 includes a body 105, an attachment 120, an applicator surface 110, and a tag 115. In some embodiments, the applicator 100 includes a cooling element 160, and one or more sensors 155A, 155B. In some embodiments, the one or more sensors 155A, 155B include a formula sensor 155A and a position sensor 115B. It should be understood that the one or more sensors 155A, 155B may be incorporated into any configuration of applicator 100, including those illustrated in FIGS. 1A-1D.

In some embodiments, the applicator includes a flat applicator surface 110. In operation, the applicator surface 110 may be moved across the skin. In some embodiments, the applicator surface includes one or more nozzles, as shown in FIG. 2B. In some embodiments, the cooling element 160 includes a Peltier plate 160. In some embodiments, the cooling element 160 is configured to pass an electric current through the Peltier plate 160 to change a surface temperature of the Peltier plate 160 and keep the Peltier plate 160 at a surface temperature.

In some embodiments, the applicator 100 further includes a formula sensor 155A and a position sensor 115B (referred to herein collectively as a position and formula deposition component). In some embodiments, the formula sensor 155A is configured to a configured to detect one or more treated areas of skin, where the one or more treated areas comprise one or more areas of the skin where the one or more formulas have been applied. In some embodiments, the formula sensor 155A is a configured to sense a reflectance of the skin. In some embodiments, the formula sensor 155A is configured to detect a hydration level, moisture level, or the like of the skin. The formula sensor 155A may be any sensor configured to determine if one or more formulas have been applied to the skin.

In some embodiments, the position sensor 155B configured to map the skin as the applicator surface applies the one or more formulas. In some embodiments, the position sensor 155A is an accelerometer, gyroscope, or the like. In some embodiments, the position sensor 155A may be an optical sensor, such as a camera. In some embodiments, the applicator 100 may include only a single sensor 155 configured to both detect whether one or more formulas have been applied to the skin and map the skin.

In operation, the position and formula deposition component is configured to sense one or more position and/or delivery measurands. The one or more position measurands include location, area dimensions, a rate of movement of the applicator, a relative distance, temperature, and the like. In some embodiments, the one or more delivery measurands include an amount of the one or more formulas deposited, a rate the one or more formulas are deposited, a frequency of depositing the one or more formulas, a pattern of the deposition of the one or more formulas, and the like. In some embodiments, the one or more measurands inform, at least partly, map data of a surface (such as the skin). In some embodiments, the one or more measurands are used by the applicator processor to generate map data and treatment area data of a skin area as the applicator surface 110 moves across the skin area and deposits the one or more formulas. In some embodiments, the treatment area data includes amount of the one or more formulas deposited, the rate of deposition of the one or more formulas, historical comparison data (i.e., one or more measurands measured during a prior application of the one or more formulas), threshold treatment data (i.e., one or more thresholds determining whether to apply the one or more formulas and/or the cooling treatment), adherence to a protocol data (i.e., how well or effectively a user is applying the one or more formulas), stop and go threshold criteria (i.e., where the one or more formulas may not be applied), and the like.

Further, in some embodiments, the cooling element 160 is operably coupled to the position and formula deposition component. The cooling element 160 may be configured to apply a cooling treatment to one or more treated areas responsive to one or more inputs from the position and formula deposition component and/or the applicator processor 140. In some embodiments, the one or more treated areas are one or more areas of the skin where the one or more formulas have been applied.

FIG. 2B is a cross section of the applicator of FIG. 2A, in accordance with the present technology. The applicator 100 may include a body 105, an applicator surface 110, an attachment 120, one or more formula reservoirs 130 configured to hold a formula F, a piston 145, and an applicator processor 140. FIG. 2B is a view taken from cross-section line C of FIG. 2A.

In some embodiments, the one or more reservoirs 130A, 130B, 130C are located inside the body 105. Each reservoir of the one or more reservoirs 130A, 130B, 130C is configured to hold one or more formulas F1, F2, F3. In some embodiments, the one or more formulas F1, F2, F3 are skin care formulas. In some embodiments, the one or more formulas F1, F2, F3 include a moisturizer, a toner, an acne treatment, a wrinkle treatment, fine line treatment, an eye cream, under eye cream, or a cosmetic. In some embodiments, the one or more formulas F1, F2, F3 are one or more ingredients of a skin care formula. In some embodiments, at least one of the one or more formulas F1, F2, F3 is a cooling formula, such as capsaicin, aloe, caffeine, or the like. As the applicator surface 110, moves, the one or more formulas F1, F2, F3 from the one or more reservoirs 130 are applied to the skin. In some embodiments, the one or more formulas F1, F2, F3 are configured to be applied under the eyes of a user. In some embodiments, each formula reservoir 130 of the formula reservoirs 130A, 130B, 130C is configured to hold a distinct formula F of a plurality of formulas F1, F2, F3. In some embodiments, each formula reservoir 130 of the plurality of formula reservoirs 130A, 130B, 130C are fluidly coupled to a nozzle 150 of a plurality of nozzles 150A, 150B, 150C on the applicator surface 110. In such embodiments, each nozzle 150 is configured to dispense a different formula F. One skilled in the art should understand that in some embodiments, a reservoir 130 of the plurality of reservoirs 130A, 130B, 130C may be fluidly coupled to more than one nozzle 150.

In some embodiments, the applicator 100 further includes a piston 145 configured to push the formula 140 towards one or more nozzles 150A, 150B, 150C as the formula is applied. In some embodiments, the piston 145 is directed by circuitry on a dispensing device or on the applicator itself to push the formula 140 towards the applicator surface.

In some embodiments, the applicator 100 includes an applicator processor 140. In some embodiments, the applicator processor 140 is configured to direct the piston 145 to push the formulas F1, F2, F3 towards the applicator surface.

FIG. 3 is an example system, in accordance with the present technology. In some embodiments, the applicator 100 can be attached to a dispensing device 200. In some embodiments, the dispensing device includes one or more light sources 220A, 220B, and an actuator 230. In some embodiments, the one or more light sources 220A, 220B may be located on the dispensing device 200, the applicator 100, or both. In some embodiments, either the dispensing device 200 or the applicator 100 includes the formula sensor 155A and/or the position sensor 155B.

In some embodiments, the dispensing device 200 includes an end 210. The end 210 may be configured to be seen through the attachment 120 on the applicator 100. In some embodiments, the end 210 includes one or more light sources 220A, 220B configured to administer light treatment to a surface while the one or more formulas and/or the cooling treatment is being applied.

In some embodiments, the one or more light sources 220A, 220B on either the dispensing device 200 or the applicator 100 are LEDs. In some embodiments, there are only two light sources 220A, 220B on the dispensing device. In some embodiments, a first light source 220A is configured to administer light therapy in a first wavelength. In some embodiments, a second light source 220B is configured to administer light therapy in a second wavelength. In some embodiments, the light therapy in the first wavelength and the light therapy in the second wavelength are administered simultaneously. In some embodiments, the light therapy, the cooling treatment, and/or applying the formula happen simultaneously.

In some embodiments, the dispensing device 200 includes one or more actuators 230. While the one or more actuators 230 is illustrated as two buttons 235, 245, in some embodiments, the one or more actuators 230 may be or include a switch, a capacitive touch type button, a dial, or the like. The one or more actuators 230 may be configured to begin the administration of light therapy, to apply the formula, or both. In some embodiments, the one or more actuators 230 include a power button 235 and an application button 245. In some embodiments, the power button 235 is configured to turn the dispensing device on or off, while the application button 245 is configured to administer one or more treatments described herein, including light treatment, cooling treatment, or the application of one or more formulas. In some embodiments, the dispensing device 200 further includes a device processor 255. In some embodiments, the device processor 255 is configured to direct the applicator 100, the dispensing device 200, or both to administer one or more treatments in response to a condition detected by the one or more sensors 135. In some embodiments, the dispensing device 200 also includes a contact-less chip reader (not pictured in FIG. 4) to read a tag on the applicator 100 (such as tag 115).

In operation, a user may place an applicator 100 into the dispensing device 200. When the actuator 230 is actuated (such as by pressing the application button 245), the formula is applied, the light therapy is administered, the cooling treatment is administered or some combination of treatments is applied, concurrently or simultaneously.

FIG. 4 is another example system, in accordance with the present technology. The system, which implements an applicator 100 and a dispensing device 200 as described herein, further includes a connected communication device 300. Optionally, the system may further include one or more external servers which are implemented as part of a cloud-computing environment.

The communication device 300 may be a personal computer (PC), a laptop computer, a PDA (Personal Digital Assistants), a smart phone, a tablet device, a UMPC (Ultra Mobile Personal Computer), a netbook, or a notebook type personal computer. In the below examples, the connected device 300 is assumed to be a smartphone, such as an Apple iphone.

The communication device 300 is capable of performing wireless communication with the dispensing device 300 by way of a wireless communication interface circuitry on the dispensing device 200 or the applicator 100. However, communication device 300 is also capable of having a wired connection to the dispensing device 200 by way of a USB interface. Additionally, the applicator 100 and the dispensing device 200 may communicate with each other and the communication device 300 through an internet connection via an 802.11 wireless connection to a wireless internet access point, or a physical connection to the internet access point, such as through an Ethernet interface. Each connected communication device 300 is capable of performing wireless communication with other devices, such as through a Bluetooth connection or other wireless means as well.

The connected communication device 300 is configured to receive information from a user for use in generating a treatment plan, including one or more treatments, that may be used by the dispenser 200 to dispense one or more formulas, one or more light treatments, or a combination thereof.

In some embodiments, the communication device 300 has a related application on it, configured to aid a user in dispensing a formula and/or applying a cooling treatment. In some embodiments, the application is configured to apply an algorithm to a photo or video of the user to detect one or more conditions. In some embodiments, the conditions may include dark circles, acne, pigmentation, rosacea, wrinkles, fine lines, or wounds. Additionally, in some embodiments, the application is configured to diagnose one or more skin conditions using an artificial intelligence (AI) algorithm. In some embodiments, the communication device 300 receives the condition detected by the one or more sensors 155A, 155B and confirms or adjusts the detected condition with an AI algorithm. In some embodiments, the communication device 300 communicates the detected condition to a user of the system and proposes one or more treatments to be applied by the applicator 100.

In some embodiments, the communication device 300 may further sense one or more environmental conditions with one or more communication device sensors. In some embodiments, the environmental conditions may include temperature, humidity, UV, pollution, and the like. In some embodiments, the application can gather environmental data from other sources such as weather services. In some embodiments, the application can further recommend a formula, comprised of one or more formulas (such as one or more skin ingredients), to the user based on the detected skin feature and/or environmental conditions.

In some embodiments, the user can set up a user profile on the application of the communication device 300. In some embodiments, setting up the user profile includes answering a user questionnaire. In some embodiments, the user questionnaire gives the user a series of inputs including past skin treatment, past use of the applicator, desired skin quality, or skin concern. In some embodiments, the application can solicit feedback from the user regarding their favorite or most effective formulation to help improve the AI algorithm.

In some embodiments, the application may direct or suggest the user apply one or more formulas, the cooling treatment, or both.

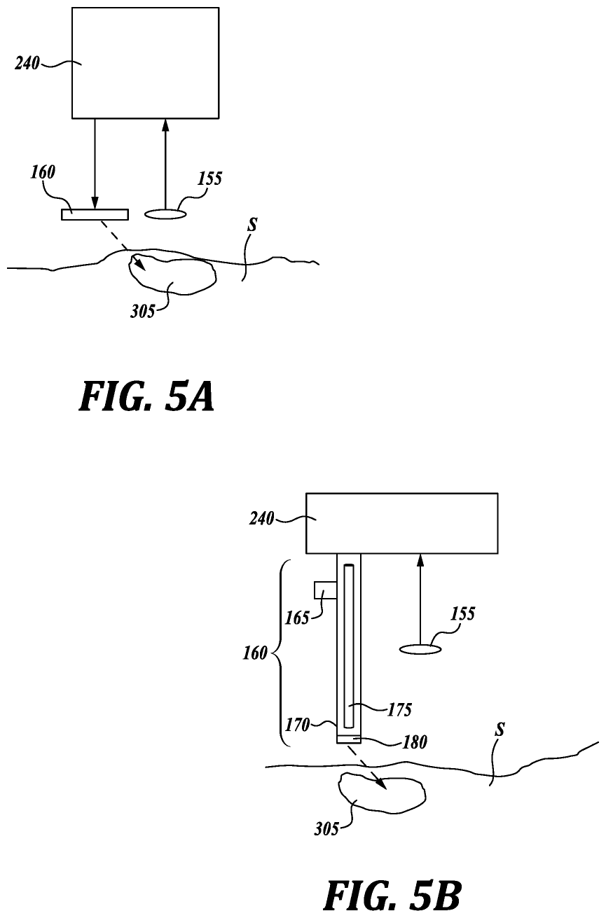

FIG. 5A is an example cooling element 160 in use, in accordance with the present technology. In some embodiments, a formula sensor 155 is configured to determine if a portion of skin S includes one or more treated areas 305. In some embodiments, a treated area 305 is an area where the one or more formulas described herein have been applied (such as formulas F1, F2, F3). In some embodiments, the formula sensor 155 transmits the detection of the one or more treated areas 305 to a processor 240. In some embodiments, the processor 240 is an applicator processor (such as applicator processor 140) or a dispenser processor (such as dispenser processor 255). In some embodiments, the processor 240 then directs the cooling element 160 to apply cooling treatment only to the one or more treated areas. In this manner, the cooling treatment may act with the one or more formulations to provide a cooling sensation to the treated portion 305 user's skin S.

FIG. 5B is another example cooling element 160 in use, in accordance with the present technology. In some embodiments, a formula sensor 155 is configured to determine if a portion of skin S includes one or more treated areas 305. In some embodiments, the formula/position sensor 155 transmits the detection of the one or more treated areas 305 to a processor 240. In some embodiments, the processor 240 is an applicator processor (such as applicator processor 140) or a dispenser processor (such as dispenser processor 255). In some embodiments, the cooling element 160 includes a pump 165, a cooling chamber 170, a solenoid 175, and an outlet 180. In some embodiments, the processor 240 then directs the cooling element 160 to apply cooling treatment only to the one or more treated areas 305. In this manner, the cooling treatment may act in tandem with the one or more formulations to provide a cooling sensation to the treated area 305 of a user's skin S.

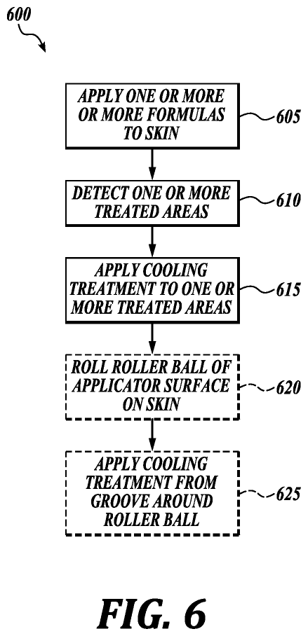

FIG. 6 is an example method 600 of using a system, in accordance with the present technology. In some embodiments, the method is applied with the system of FIG. 3 or FIG. 4.

In block 605, one or more formulas are applied to a user's skin. In some embodiments, the one or more formulas are applied with an applicator (such as applicator 100). In some embodiments, a processor (such as applicator processor 140, dispenser processor 255, or a combination thereof) directs the applicator to apply the one or more formulas.

In block 610, one or more treated areas (such as treated area 305) are detected. As described herein, a treated area is any area of the skin where the one or more formulas have been applied. In some embodiments, the one or more treated areas may be detected by a sensor on the applicator (such as formula sensor 155). In some embodiments, the one or more treated areas may be detected by the position and formula deposition component as described herein.

In block 615, cooling treatment is applied to the one or more treated areas. In some embodiments, the cooling treatment is applied with a cooling element, such as the cooling element 160 of FIG. 1B or FIG. 2B. In some embodiments, the cooling element is a Peltier plate configured to apply a cooling treatment to the one or more treated areas. In some embodiments, the cooling element includes a pump, a cooling chamber, a solenoid, and an outlet. In some embodiments, the cooling treatment is applied around or on an applicator surface, such as applicator surface 110 of FIG. 1B or FIG. 2B.

Optionally, in block 620, when the applicator surface is a roller ball, the roller ball is rolled onto the skin. In some embodiments, as the roller ball rolls, the one or more formulas and/or the cooling treatment is applied.

Optionally, in block 625, cooling treatment is applied from a groove located around the roller ball as described in FIG. 1D. In some embodiments, the groove is configured to administer cooling treatment in the form of cool air in 360 degrees from the applicator surface. In some embodiments, the cooling treatment is directed to flow from only a portion of the groove. For example, in some embodiments, the cooling element is configured to administer cooling treatment in the direction that the applicator 100 is being moved about the skin.

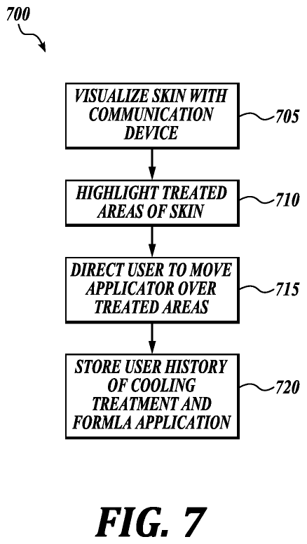

FIG. 7 is another example method of using a system in accordance with the present technology. In some embodiments, the method 700 is carried out by a communication device (such as communication device 300). In some embodiments, the method 700 is carried out by a dispensing device (such as the dispensing device 200). In some embodiments, the method 700 may be a part of the method 600.

In block 705, the skin is visualized. In some embodiments, the skin is visualized by providing a graphic or a live feed on the communication device. In some embodiments, a position sensor (such as position sensor 155B) includes a camera, configured to take one or more images (i.e., photos, videos, or live feeds) of the skin as the user moves the applicator over the skin. In some embodiments, the skin is visualized with a camera on the communication device. In some embodiments, the skin is visualized by the position and formula deposition component as described herein.

In block 710, one or more portions of the skin are highlighted. In some embodiments, the one or more portions are highlighted on the communication device, either graphically, or overlaid on an image taken with the one or more sensors. In some embodiments, the one or more portions are one or more treatment areas, as described herein, where formula has already been applied. In some embodiments, the one or more portions are untreated areas, as in, where formula has not been applied. In some embodiments, both treated areas and untreated areas are highlighted. In some embodiments, the one or more portions are areas of interest. In some embodiments, different colors, symbols, patterns, or the like may be used to distinguish the areas of interest. For example, one or more portions of skin where formula has been applied (treated areas) may be demarcated with red patches, and one or more portions of the skin where formula has not been applied (untreated areas) may be demarcated with yellow patches. One skilled in the art will appreciate that the areas of interest may be highlighted in any manner.

In block 715, the communication device or dispensing device may direct the user to move the applicator over the one or more portions. In some embodiments, as the applicator moves over the skin, the one or more treatments (such as formula application or cooling treatment) can be changed to adapt to each of the areas of interest. For example, in some embodiments, when the applicator moves over an untreated area, the applicator may be directed to apply one or more formulas. In some embodiments, when the applicator moves over a treated area, the application is directed to apply the cooling treatment.

In block 720, the communication device or the dispensing device stores a user history. As described herein, the user history may include a history of the one or more treatments, the location where one or more formulas and/or the cooling treatment has been applied, the location where one or more formulas and/or the cooling treatment has been applied, a condition of one or more portions of the skin, and the like.

It should be understood that methods 600 and 700 should be interpreted as merely representative. In some embodiments, process blocks of methods 600 and 700 may be performed simultaneously, sequentially, in a different order, or even omitted, without departing from the scope of this disclosure.

The present application may reference quantities and numbers. Unless specifically stated, such quantities and numbers are not to be considered restrictive, but representative of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

While illustrative embodiments have been illustrated and described, it will be appreciated that various changes can be made therein without departing from the spirit and scope of the invention.

The embodiments of the invention in which an exclusive property or privilege is claimed are defined as follows:

1. An applicator, comprising:
a position and formula deposition component configured to sense position measurands (including location, area dimensions, rate of movement, relative distance, temperature etc.) and delivery measurands (including amount deposited, rate of deposition, frequency of deposition, deposition pattern, etc.) and to generate map data and treatment area data of a skin area (including, amount deposited, rate of deposition, historical comparison data, threshold treatment data, adherence to a protocol data, stop and go threshold criteria, etc.) as an applicator surface applies one or more formulas to the skin area; and
a cooling element operably coupled to the position and formula deposition component, the cooling element configured to apply a cooling treatment to one or more treated areas responsive to one or more inputs from the position and formula deposition component wherein the one or more treated areas are one or more areas of the skin where the one or more formulas have been applied.

2. The applicator of claim 1, further comprising:
an applicator surface configured to apply one or more formulas to skin; and
one or more reservoirs configured to hold the one or more formulas.

3. The applicator of claim 1, wherein the cooling element comprises a Peltier plate.

4. The applicator of claim 1, wherein the cooling element comprises:
a pump configured to pump air;
a compression chamber coupled to the pump, wherein the compression chamber is configured to move the air to the applicator surface;
a solenoid configured to move the air towards the outlet; and
an outlet configured to cool and apply the air to the skin.

5. The applicator of claim 1, wherein the applicator is configured to direct the cooling element to apply the cooling treatment only to the one or more treated areas.

6. The applicator of claim 1, wherein the applicator surface comprises one or more nozzles, wherein the one or more nozzles are in fluid communication with the one or more reservoirs.

7. The applicator of claim 6, wherein each nozzle of the one or more nozzles is configured to dispense a different formula of the one or more formulas.

8. The applicator of claim 1, wherein the applicator surface is a roller ball, wherein the roller ball is in fluid communication with the one or more reservoirs and the roller ball is configured to apply the one or more formulas as the roller ball rolls.

9. The applicator of claim 8, wherein the applicator further comprises a groove located around the roller ball.

10. The applicator of claim 9, wherein the cooling element is configured to apply the cooling treatment from the groove on the applicator.

11. A system for applying a cooling treatment, the system comprising:
the applicator of claim 1, and
a dispensing device configured to couple to the applicator, wherein the dispensing device comprises:
a processor configured to direct the applicator to apply the one or more formulas.

12. The system of claim 11, wherein the applicator surface is a roller ball, wherein the roller ball is in fluid communication with the one or more reservoirs and the roller ball is configured to apply the one or more formulas as the roller ball rolls.

13. The system of claim 12, wherein the applicator further comprises a groove located around the roller ball.

14. The system of claim 13, wherein the cooling element is configured to apply the cooling treatment from the groove on the applicator.

15. The system of claim 11, wherein the cooling element comprises a Peltier plate.

16. The system of claim 11, wherein the cooling element comprises:
a pump configured to pump air;
a compression chamber coupled to the pump, wherein the compression chamber is configured to move the air to the applicator surface;
a solenoid configured to move the air towards the outlet; and
an outlet configured to cool and apply the air to the skin.

17. The system of claim 11, wherein the formula sensor is further configured to transmit treatment area data to the processor of the applicator, and wherein the processor is further configured to direct the applicator to apply a cooling treatment to the one or more treated areas.

18. The system of claim 11, wherein the system further comprises a communication device, configured to:
visualize the skin;
highlight one or more treated areas of the skin, wherein the one or more treated areas of the skin are areas on the skin where a user has applied the one or more formulas, locations based on a user history, or a combination thereof;
direct the user to move the applicator over the one or more treated areas; and
store the user history, wherein the user history is each time, location, or a combination thereof that the user applies the cooling treatment, the formula, or a combination thereof.

19. A method of applying a cooling treatment with the system of claim 11, the method comprising:
applying one or more formulas to skin;
detecting one or more treated areas, wherein the one or more treated areas comprise areas of the skin where the one or more formulas have been applied; and
applying a cooling treatment to the one or more treated areas with the cooling element.

20. The method of claim 19, wherein the method further comprises:
rolling a roller ball of the applicator surface on the skin to apply the one or more formulas to the skin; and
applying the cooling treatment from a groove around the roller ball.

21. The method of claim 19, wherein the method further comprises:
visualizing the skin with a communication device;
highlighting one or more treated areas of the skin, wherein the one or more treated areas of the skin are areas on the skin where a user has applied the one or more formulas, locations based on a user history, or a combination thereof;
directing the user to move the applicator over the one or more treated areas; and
storing the user history, wherein the user history is each time, location, or a combination thereof that the user applies the cooling treatment, the formula, or a combination thereof.
